(19) United States
(12) Patent Application Publication
(10) Pub. No.: US 2022/0296409 A1
(43) Pub. Date: Sep. 22, 2022

(54) APPLICATOR FOR A MENSTRUAL DEVICE

(71) Applicant: The Flex Company, Venice, CA (US)

(72) Inventors: Andrew Ross Miller, Oakland, CA (US); Lauren Schulte Wang, Topanga Canyon, CA (US)

(21) Appl. No.: 17/701,530

(22) Filed: Mar. 22, 2022

Related U.S. Application Data
(60) Provisional application No. 63/164,267, filed on Mar. 22, 2021.

(51) Int. Cl. A61F 5/455; A61F 5/44

(52) U.S. Cl. CPC A61F 5/4553; A61F 5/4404

(57) ABSTRACT

A menstrual disc applicator that is configured to hold a menstrual device in a folded configuration to allow for insertion of the disc into the fornix. Many applicators are configured to not only insert the disc but to provide rotation such that it sits properly in the vaginal fornix. Some applicators may have spring like mechanisms to help move the disc into the proper position while others may have contoured elements that better conform to the anatomy to allow for proper insertion and placement of the disc.

### CROSS-REFERENCE TO RELATED APPLICATIONS

This application claims the benefit of U.S. Provisional Application No. 63/164,267, filed Mar. 22, 2021.

### FIELD OF THE INVENTION

This matter generally relates to applicators for menstrual devices, and more particularly to an applicator for a menstrual disc.

### BACKGROUND OF THE INVENTION

Tampons are less bulky than pads and are thus a popular feminine hygiene product. But tampons are associated with various negative health issues such as toxic shock syndrome. In addition, tampon usage raises waste disposal concerns. Menstrual cups and menstrual discs offer the comfort advantage of tampons without the health risks and waste disposal issues. A menstrual cup is typically bell-shaped with a diameter of, for example, approximately 1.5 inches. In contrast, a menstrual disc is markedly wider, for example having a width of approximately 2.5 inches. The size difference between menstrual discs and menstrual cups relates to their different applications. A menstrual cup is designed to fit somewhat low in the vaginal canal whereas a menstrual disc sits much higher, typically in the vaginal fornix about the cervix.

To seal about the cervix in the vaginal fornix, a menstrual disc includes a relatively robust and resilient rim. In contrast, a catchment portion of the menstrual disc may be a thin-film material. Given the relatively large diameter of the rim, a menstrual disc cannot be inserted unfolded into the vaginal canal. Instead, a user folds or collapses the disc into an elongated “FIG. 8” configuration. The folded rim may then be more easily inserted into the vaginal canal and guided back to the fornix. The menstrual disc then unfolds due to the resilient rim and seals about the cervix. Due to the disc location in the fornix as compared to the cup location in the vaginal canal, a menstrual disc may exert less pressure and thus be more comfortable. In addition, a menstrual disc can generally hold more menses and accommodate intercourse.

Despite the advantages of a menstrual disc over a menstrual cup, there are some impediments to the use of a menstrual disc. For example, the cervix is not aligned with the vaginal canal but instead is tilted approximately 90 degrees with respect to the vaginal canal longitudinal axis. To fit into the vaginal fornix about the cervix, the menstrual disc must rotate due to the tilting of the cervix with respect to the vaginal canal. In particular, a distal end of the folded disc must rotate downwards as the folded disc is inserted towards a distal end of the vaginal canal whereas a proximal end of the folded disc must rotate upwards with this insertion as the folded disc unfolds and seals about the cervix. There is thus a need in the art for menstrual discs with improved insertion features.

### SUMMARY

In accordance with one aspect of the disclosure, a menstrual disc applicator is provided that includes: a housing having a lumen extending a length from a proximal end of the housing to an open distal end, the lumen having a diameter sufficient to receive a proximal portion of a folded menstrual disc and having a length that is less than a length of the folded menstrual disc; and a plunger configured to engage within the lumen.

In accordance with another aspect of the disclosure, a menstrual disc applicator system is provided that includes: a folded menstrual disc having a proximal portion and a distal portion; and a housing having a lumen extending a length from a proximal end of the housing to an open distal end, wherein the proximal distal portion of the folded menstrual disc is received by the lumen and the distal portion of the folded menstrual disc distally extends from the open distal end of the housing.

In accordance with yet another aspect of the disclosure, a method of inserting a menstrual disc is provided that includes the acts of: inserting a folded menstrual disc into a lumen of an applicator so that a proximal end of a proximal portion of the folded menstrual disc is substantially aligned with a proximal end of a housing of the applicator and so that a distal portion of the folded menstrual disc protrudes from an open distal end of the housing of the applicator to provide a disc-loaded applicator; inserting the disc-loaded applicator into a vaginal canal; and pushing a plunger of the disc-loaded applicator to expel the folded menstrual disc into the vaginal canal so that the folded menstrual disc unfolds and rotates into position in a vaginal fornix to seal about a cervix.

### BRIEF DESCRIPTION OF THE DRAWINGS

The description will be more fully understood with reference to the following figures, which are presented as exemplary embodiments of the disclosure and should not be construed as a complete recitation of the scope of the claimed subject matter, wherein:

FIG. 1 illustrates a cross sectional view of a menstrual disc being placed into the anatomically correct position.

FIG. 2 illustrates an applicator device for a menstrual disc in accordance an embodiment of the disclosure.

FIG. 3 illustrates an applicator device receiving a folded menstrual disc to form a menstrual disc applicator system in accordance with an embodiment of the disclosure.

FIGS. 4A and 4B illustrate an applicator device with a hooked plunger in accordance with embodiments of the disclosure.

FIGS. 5A and 5B illustrate an applicator device with a beveled plunger in accordance with embodiments of the disclosure.

FIGS. 5A and 5B illustrate an alternative embodiment of an applicator.

FIGS. 6A and 6B illustrate an applicator device designed for passive insertion in accordance with embodiments of the disclosure.

FIGS. 7A and 7B illustrate an applicator with internal configuration elements in accordance with embodiments of the disclosure.

FIGS. 8A and 8B illustrate various rims of menstrual discs in accordance with embodiments of the disclosure.

FIG. 9 illustrates various rims of menstrual discs with ridges and/or grooves to aid in reducing friction between the menstrual discs and the walls of the applicator housing lumen in accordance with an aspect of the disclosure.

### DETAILED DESCRIPTION OF THE INVENTION

A menstrual disc applicator is provided that solves the insertion issues of a menstrual disc with regard to rotating the menstrual disc into the proper position in the fornix. Some embodiments may be designed for other items designed to be inserted into the vaginal canal such as diaphragms, suppositories, tools etc. Additionally, the applicator can have a number of different types of mechanisms that can push, position, and/or release a menstrual disc such that it is positioned in the anatomically correct position against the cervix.

Traditional feminine hygiene applicators have been primarily designed for use with tampons. However, such applicators are not readily applicable for use with menstrual discs. For example, a tampon length is relatively small compared to a menstrual disc width. Given this relatively short length, a tampon may thus be completely contained within a tampon applicator prior to insertion. But a tampon applicator is not suitable for menstrual discs. For example, a tampon applicator will typically have a rounded end formed by several leaves or petals. As a user pushes the tampon applicator plunger, the leaves bend outward so that the tampon may be expelled from the applicator. But a tampon is a relatively dense woven product whereas a menstrual disc rim is resilient. A menstrual disc thus cannot be readily plunged down the bore of an applicator to bend any leaves or petals at the distal end of the applicator. More fundamentally, a tampon-style applicator will simply push the disc into the distal end of the vaginal canal without the necessary rotation to rotate the disc into position about the cervix. In addition, a tampon-style applicator would need to be relatively long due to contain the folded disc. A user would then have difficulty holding the applicator in position while attempting to push the applicator plunger due to the relative length of the combination of the withdrawn plunger and the applicator body. These issues are solved by the menstrual disc applicator disclosed herein.

To provide a menstrual applicator that readily provides the proper rotation to the menstrual disc, a hollow applicator body is disclosed having a length that is shorter than a length of the folded disc. The applicator body has a lumen for a portion of the folded disc. The lumen extends from an open proximal end for receiving the plunger and an open distal end for delivering the folded disc. Due to the shorter length of the applicator body, the lumen can accommodate just a portion of the folded menstrual disc. When the folded menstrual disc is inserted into the lumen of the applicator body, a distal portion of the folded menstrual disc thus protrudes from the distal end of the applicator body.

Due to the resilient rim, a menstrual disc will develop a mild arc or rainbow shape along its length when folded. The distal portion of the folded menstrual disc that protrudes from the distal end of the applicator body will thus angle downwardly with respect to a longitudinal axis of the applicator body. This is quite advantageous in satisfying the downward rotation on the distal end of the folded disc that is needed during insertion. To assist in the upward rotation on the proximal end of the folded disc that is also needed during insertion, the open distal end of the applicator housing is beveled akin to the beveling of a hypodermic needle except that the tip of the bevel is rounded. The beveling is aligned with the downward arching of the folded menstrual disc such that the rounded tip of the open distal end of the applicator housing faces or supports a proximal end of the protruding distal portion of the folded disc. The rounded tip of the open distal end thus forms a beak or extension relative to an upper edge of the bevel. This is quite advantageous in that as the folded disc is extruded past the bevel, there is no upper edge of the applicator housing above the rounded tip of the open distal end. The proximal edge of the folded disc will thus tend to be rotated upwardly as the folded disc is extruded from the applicator housing into the vaginal fornix.

The use of an applicator is familiar to tampon users. This familiarity may then be applied to the menstrual disc applicator disclosed herein. In addition, a user may be hesitant with regard to hand placing a menstrual disc given the presence of menses in the vaginal canal. An applicator thus offers improved hygiene to such users.

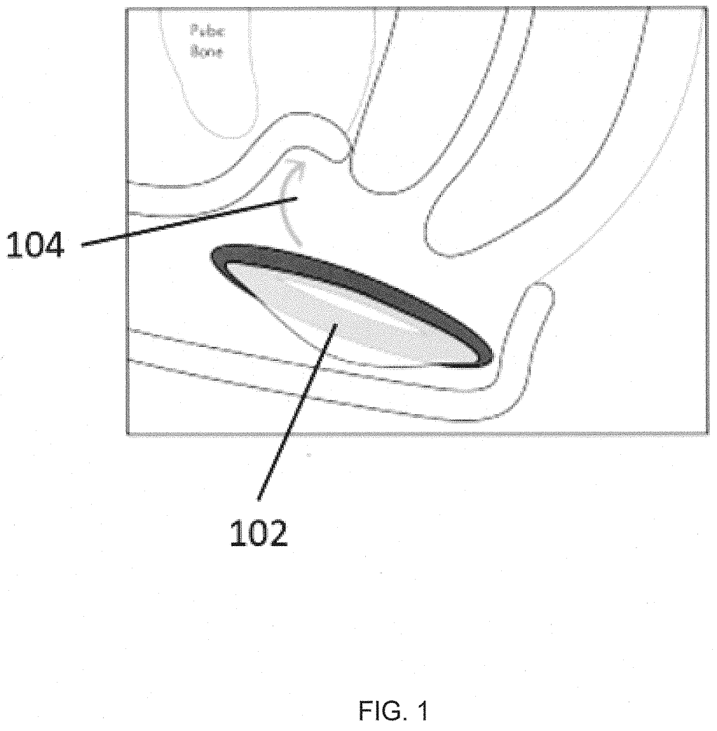

The rotation of a menstrual disc into position in the vaginal fornix by the applicator disclosed herein may be better appreciated with reference to FIG. 1. Due to the downward angled bias from the applicator, a distal end of a menstrual disc has passed the protruding cervix and has engaged with the posterior side of the vaginal fornix. As will be explained further herein, menstrual disc 102 when folded and inserted into the applicator body lumen (not illustrated) is arched so as to be convex with respect to the floor of the vaginal canal. Due to this arch, a proximate end of menstrual disc 102 will tend to rotates upwardly as indicate by arrow 204 into an anterior portion of the vaginal fornix. This upward rotation of the proximate end of menstrual disc 102 is aided by an open distal end of the applicator body as will be explained further herein. After upward rotation 204, menstrual disc is sealed about the cervix and abuts the full circumference of its rim with the vaginal fornix.

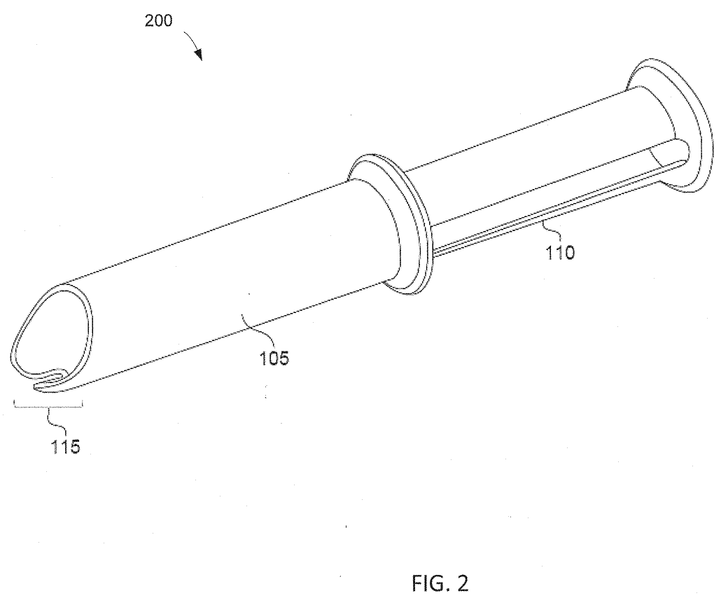

An example menstrual disc applicator 200 will now be discussed as shown in FIG. 2 that assists in the rotation of the menstrual disc as it unfolds and is extruded from applicator 200 into the vaginal fornix. Applicator 200 includes an applicator body 105 having a lumen extending from a beveled distal end 115 to a flanged proximal end 120. A plunger 110 engages the lumen of applicator body 105. As shown in FIG. 2, plunger 110 is in a withdrawn position so that a folded menstrual disc may be loaded into the applicator body lumen.

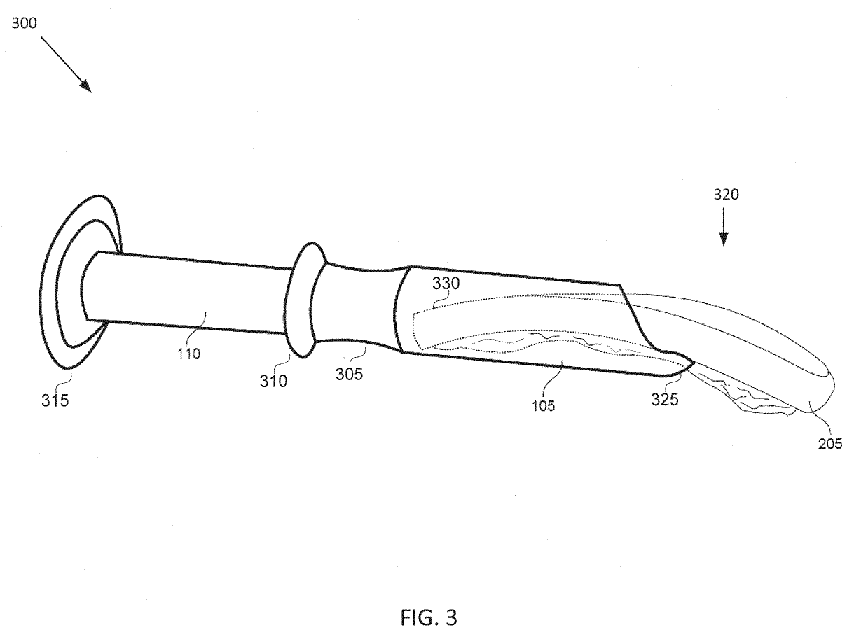

Another menstrual disc applicator 300 is shown in FIG. 3 with a folded menstrual disc 205 loaded into the applicator body lumen. Applicator body 105 joins to a narrowed proximal portion 305 having a flange 310 for engaging with a corresponding flange 315 on plunger 110. Due to narrowed proximal portion 305, folded menstrual disc 205 is contained only within applicator body 105. A length of applicator body is shorter than a length of folded menstrual disc such that a distal portion 320 of folded menstrual disc 205 extends beyond a rounded tip 325 at the distal end of applicator body 105. Due to the folding, folded menstrual disc 205 has an arched shape that arches up to a high point near an upper edge of beveled distal end 115 (FIG. 2). The beveling at the distal end of applicator body 105 is aligned with the downward arching of the folded menstrual disc 205 such that abuts a proximal end 330 of folded menstrual disc 205 as the proximal end 330 is finally extruded from applicator body 105 due to a user pushing flanges 315 and 310 together. As noted earlier, this is quite advantageous in that there is no upper edge of the applicator housing directly above the rounded tip 325 due to the beveling. The arching of the menstrual disc 205 provides the downward rotation of the distal end of menstrual disc 205 whereas this arching and the open upper edge of the applicator housing 105 provides the upward rotation of the proximal end of menstrual disc 205. Due to the relative shortness of applicator housing 105, a user may readily hold applicator 300 in one hand as they would a tampon applicator during the menstrual disc insertion.

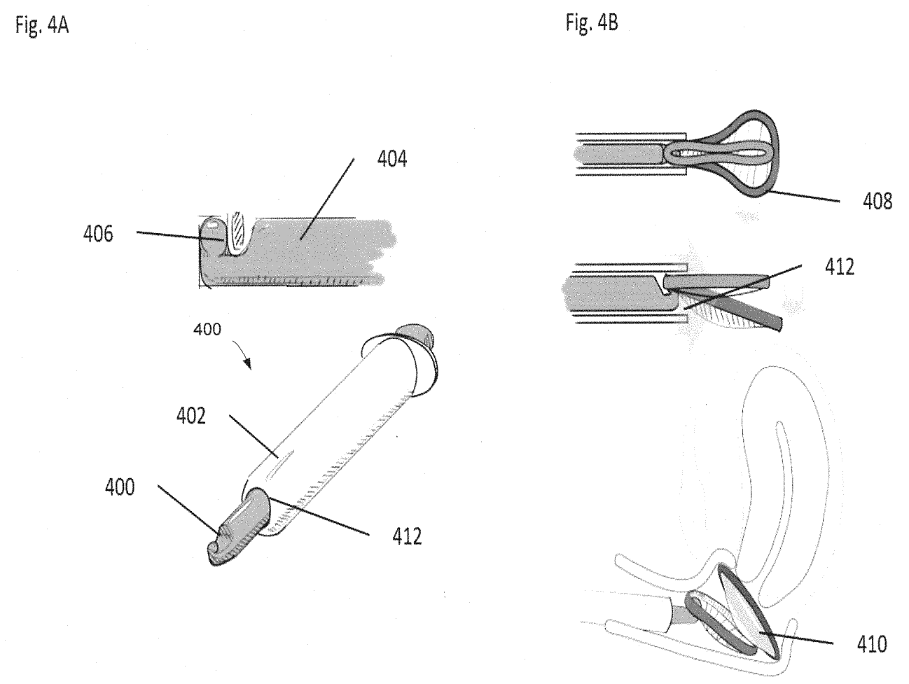

It will be appreciated that various modifications may be made to the applicators disclosed herein. For example, FIGS. 4A and 4B illustrate a menstrual applicator 400 in which a plunger 404 has a distal hooked end 406 that is designed to engage with a rim 408 of a menstrual disc 410. Applicator 400 includes an applicator housing 402 with a lumen for receiving plunger 404. Advantageously, a user may engage rim 403 with hooked end 406 to draw the disc 410 into distal opening 412 of housing 402. As the disc 410 is drawn into the lumen of applicator housing through the opening 412, disc 410 folds in a manner that allows for improved insertion. This may be better illustrated in FIG. 4B. In addition to the ability to grasp and fold the disc 410 using hooked end 406, the applicator 400 can be configured for improved insertion of the disc 410 as the plunger 404 is pushed into applicator housing 402 towards distal opening 412.

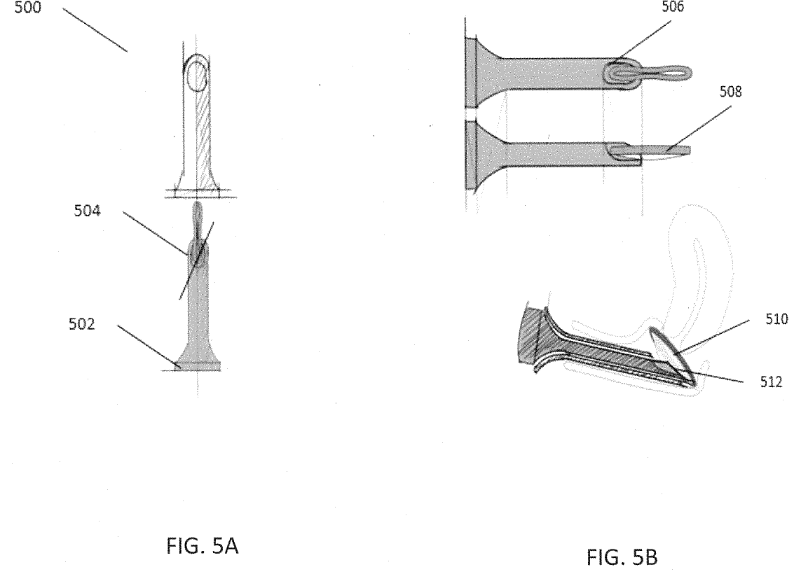

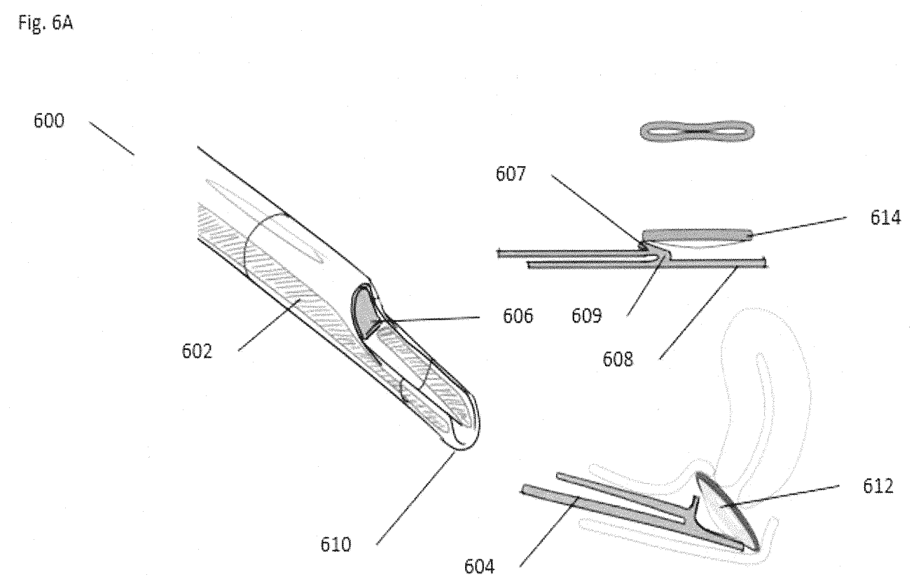

Other embodiments may include a plunger element with a distal end that is beveled similarly to the beveling at the distal end of the applicator housing. Such a beveled distal end of the plunger aids in the desired rotation of the menstrual disc during insertion. For example, FIGS. 5A and 5B illustrate a plunger 500 with a flanged proximal end 502 and a beveled distal end 504. Beveled distal end 504 has a contoured portion 506 or cutout that engages with a rim 508 of a menstrual disc 510. The contoured portion 506 can hold the disc 510 in place during insertion and then can provide an angled surface 512 that can be used to push the disc 510 further into the proper insertion angle.

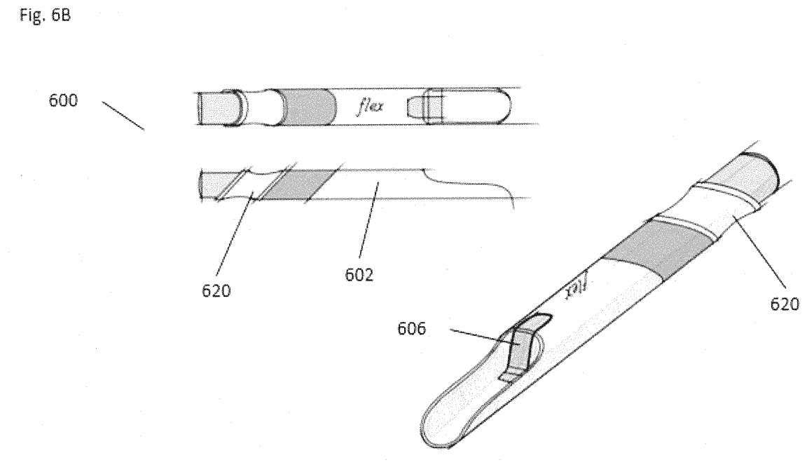

In alternative implementations, applicators can be configured to push and eject the menstrual disc such that it is mechanically positioned by the plunger itself. For example. FIGS. 6A and 6B illustrate an applicator 600 with an applicator housing 602 and a plunger 604 that includes a resilient element 606 that functions similarly to a kickstand. The resilient element 606, or kickstand, can be formed from a protrusion 607 extending off of a base element 608 of plunger 604 and further connected to a tension or force element 609. When the plunger 604 is positioned within the housing 602 the resilient element 606 is held under pressure from the housing 602. This stored energy can then be used to move the kickstand 606 into position when the plunger 604 is moved towards a distal end 610 of the housing 602.

In many embodiments, the distal end 610 of the housing 602 can be configured to hold or secure a menstrual disc 612 such that a portion of a rim 614 of the disc 612 rests against the kickstand 606. The holding mechanism can be a channel with some type of gripping element that passively holds the disc 612. As the plunger is moved towards the distal end 610 the release of energy from the compression of kickstand 606 can force the disc 612 into the proper position in the fornix such that the disc 612 will seal about the cervix.

Since the insertion of the disc can pose some potential dexterity issues, it can be appreciated that some embodiments may incorporate one or more different features that can improve the ergonomics of the applicator. For example, FIG. 6B illustrates an embodiment of applicator 600 having the kickstand element 606 in which the housing 602 has one or more contoured surfaces 620 that can allow for improved grip of the user. As can be appreciated, contoured surfaces can improve the ergonomics of the device and ultimately improve usability. A similar contoured surface is provided by narrowed proximal portion 305 adjoining applicator housing 105 in applicator 300 of FIG. 3.

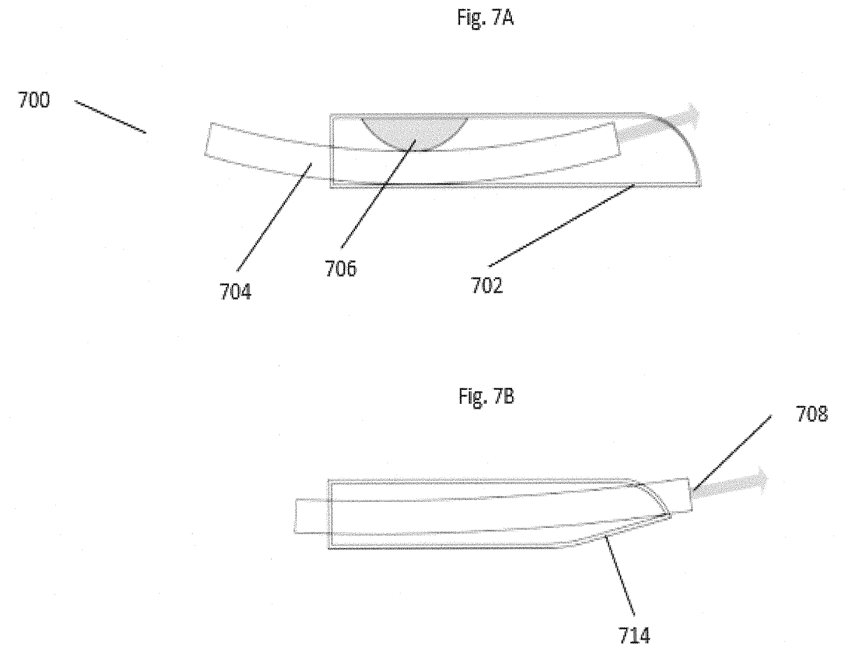

Additionally, some embodiments may incorporate one or more internal elements within a housing element to help ensure the proper angle for insertion is achieved by the applicator. As an example, FIGS. 7A and 7B illustrate cross sectional views of an applicator 700 with a housing 702 and a plunger 704. FIG. 10A illustrates a cam 706 within the housing 702. The cam 706 engages the plunger 704 so that it curves the plunger 704 and thus bends a distal end of plunger 704 with respect to the housing 702. This can be highly beneficial because it can angle the disc (not illustrated) at the appropriate insertion angle for placement in the fornix. Further, the disc may be partially or completely contained within the applicator before the plunger 704 is advanced, and in the partially inserted case, the curvature of the disc may further aid in providing the requisite angle for proper delivery in the fornix. Likewise, FIG. 7B illustrates an alternative embodiment of applicator 700 with an angled side 714 of the housing 702. The angled side 714 or portion of the housing 702 can act in a similar manner as the cam 706 such that the plunger is moved or directed towards the proper angle for insertion.

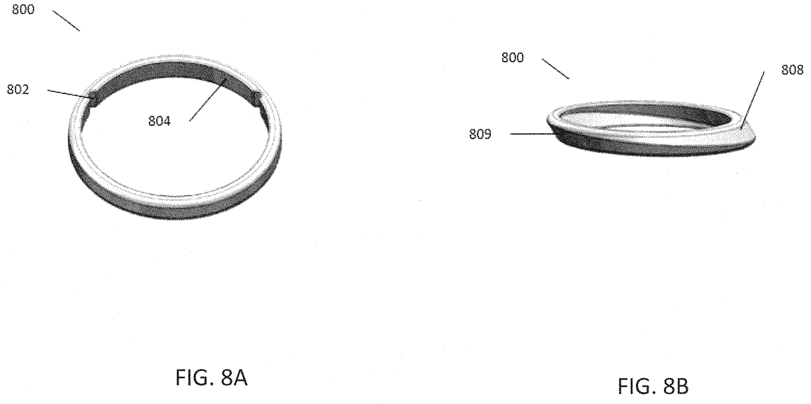

To aid in the folding of the menstrual disc so that it may be partially contained within the applicator housing, a rim 800 of a menstrual disc may include notches 802 on the interior wall 804 of rim 800 as shown in FIG. 8A. Notches 802 may be located so that one notch is at the proximal end of the folded menstrual disc and another at the distal end of the folded menstrual disc. A side 808 of the menstrual disc rim 800 that forms the distal end when folded may be beveled to aid insertion as shown in FIG. 8B. An opposing side 809 has a reverse bevel to aid in engagement of the plunger.

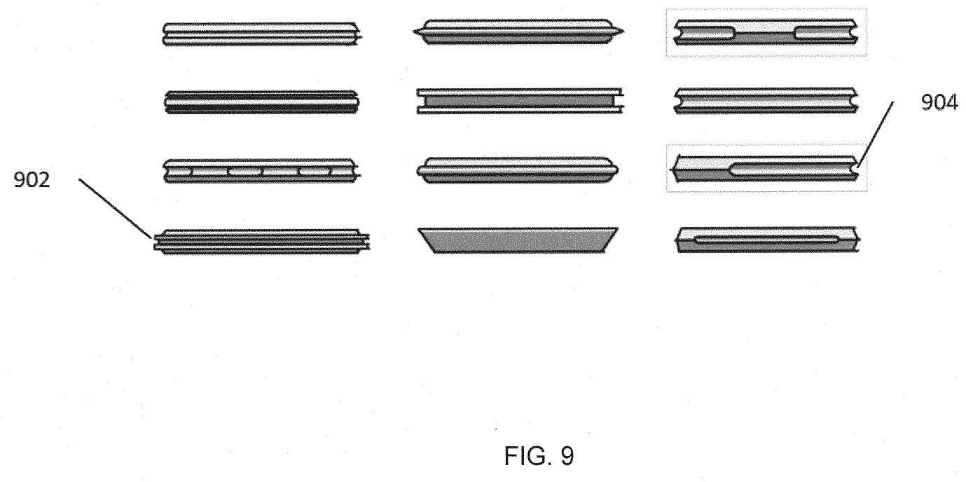

To reduce friction with the walls of the applicator body lumen, the menstrual disc rim may include a variety of ridges 902 or grooves 904 as shown in FIG. 9. In this fashion, the surface area of the menstrual disc rim that contacts the walls of the applicator body lumen is reduced.

As has been described above, many discs or disc rims can have a number of different features to help improve the insertion of the disc and ensure the disc is inserted at the proper angle and location. Accordingly, applicators can have similar features that engage with one or more portions of the rim to help secure the disc for insertion as well as ensure the disc is placed at the proper location and/or angle.

### DOCTRINE OF EQUIVALENTS

As can be inferred from the above discussion, the above-mentioned concepts can be implemented in a variety of arrangements in accordance with embodiments of the invention. Specifically, an applicator for holding and inserting a menstrual disc such that the disc can be inserted at the proper position within the fornix. Achieving such functionality, according to embodiments, involves the implementation of special arrangements/designs between subsystems described above, and their equivalents.

Accordingly, although the disclosure has been described in certain specific aspects, many additional modifications and variations would be apparent to those skilled in the art. It is therefore to be understood that the claimed subject matter may be practiced otherwise than specifically described. Thus, embodiments of the present disclosure should be considered in all respects as illustrative and not restrictive.

What is claimed is:

1. A menstrual disc applicator comprising:
a housing having a lumen extending a length from a proximal end of the housing to an open distal end, the lumen having a diameter sufficient to receive a proximal portion of a folded menstrual disc and having a length that is less than a length of the folded menstrual disc, wherein the open distal end is beveled from an extending bottom portion to a recessed upper portion; and
a plunger configured to engage within the lumen.

2. The menstrual disc applicator of claim 1, wherein the open distal end is beveled from an extending bottom portion of the open distal end to a recessed upper portion of the open distal end.

3. The menstrual disc applicator of claim 2, wherein the extended bottom portion of the open distal end is rounded.

4. The menstrual disc applicator of claim 1, wherein the plunger includes a plunger flange at a distal end of the plunger.

5. The menstrual disc applicator of claim 4, further comprising a housing flange at the proximal end of the housing, wherein the housing flange is configured to engage with the plunger flange when the plunger in an extended position in the lumen.

6. The menstrual disc applicator of claim 1, wherein the lumen is narrowed at the proximal end of the housing.

7. A menstrual disc applicator system, comprising:
a folded menstrual disc having a proximal portion and a distal portion; and
a housing having a lumen extending a length from a proximal end of the housing to an open distal end, wherein the proximal distal portion of the folded menstrual disc is received by the lumen and the distal portion of the folded menstrual disc distally extends from the open distal end of the housing.

8. The menstrual disc applicator system of claim 7, further comprising:
a plunger configured to engage with the lumen and to extrude the folded menstrual disc from the open distal end of the housing.

9. The menstrual disc applicator system of claim 7, wherein the wherein the open distal end is beveled from an extending bottom portion to a recessed upper portion.

10. The menstrual disc applicator system of claim 9, wherein the extending bottom portion is rounded.

11. The menstrual disc applicator system of claim 7, wherein a rim of the folded menstrual disc has an inner surface having a first notch at a proximal end of the proximal portion and having a second notch at a distal end of the distal portion.

12. The menstrual disc applicator system of claim 7, wherein a rim of the folded menstrual disc is beveled to aid insertion into the vaginal canal.

13. The menstrual disc applicator system of claim 8, wherein a distal end of the plunger includes a hook configured to engage with a rim of the folded menstrual disc.

14. The menstrual disc applicator system of claim 7, wherein the folded menstrual disc is arched so that a midpoint of the folded menstrual disc engages with an upper surface of the lumen.

15. The menstrual disc applicator system of claim 14, wherein the folded menstrual disc is further arched so that a distal end of the folded menstrual disc engages with a lower surface of the lumen.

16. The menstrual disc applicator system of claim 14, wherein a rim of the folded menstrual disc includes at least one ridge.

17. The menstrual disc applicator system of claim 14, wherein a rim of the folded menstrual disc includes at least one groove.

18. A method of inserting a menstrual disc comprising:
inserting a folded menstrual disc into a lumen of an applicator so that a proximal end of a proximal portion of the folded menstrual disc is substantially aligned with a proximal end of a housing of the applicator and so that a distal portion of the folded menstrual disc protrudes from an open distal end of the housing of the applicator to provide a disc-loaded applicator;
inserting the disc-loaded applicator into a vaginal canal; and
pushing a plunger of the disc-loaded applicator to expel the folded menstrual disc into the vaginal canal so that the folded menstrual disc unfolds and rotates into position in a vaginal fornix to seal about a cervix.
